(19) United States
(12) Patent Application Publication
(10) Pub. No.: US 2007/0055084 A1
(43) Pub. Date: Mar. 8, 2007

(54) Method for producing haloalkanes from alcohols

(75) Inventors: Viet Stegmann, Mannhiem (DE); Klemens Massonne, Bad Durkheim (DE)

Correspondence Address: CONNOLLY BOVE LODGE & HUTZ, LLP, P O BOX 2207, WILMINGTON, DE, 19899, US

(73) Assignee: BASF Aktiengesellschaft, Ludwigshafen (DE)

(21) Appl. No.: 10/570,765

(22) PCT Filed: Sep. 7, 2004
(86) PCT No.: PCT/EP04/09974

§ 371 Date: Sep. 29, 2006

(30) Foreign Application Priority Data
Sep. 8, 2003 (DE) 103 41 308.1

(51) Int. Cl. C07C 15/12

(52) U.S. Cl. 585/25

(57) ABSTRACT

The invention relates to a process for preparing haloalkanes by reaction of alcohol with hydrogen halide, wherein the reaction of the alcohol with the hydrogen halide occurs in the presence of an ionic liquid at a temperature which is above 100° C. for at least part of the time and, at least at the time of the commencement of the reaction, the water content is not more than 25 mol % based on the amount of liquid, where the ionic liquid is not octyltrimethylammonium chloride.

The present invention relates to a process for preparing haloalkanes by reacting alcohols with hydrogen halide in the presence of an ionic liquid.

The process corresponds to the following general reaction equation, where R is any alkyl radical
R—OH+HX→R—X+H₂O.

A large number of processes for the halogenation of alcohols by hydrogen halides are described in the literature. In these processes, the reaction is carried out in the presence of an aqueous base (usually alkylamines or pyridine derivatives or their salts), with the base functioning as catalyst.

EP-A 0 428 166 describes a process for preparing haloalkanes having from 1 to 4 carbon atoms in the presence of an amine hydrohalide. As parent amine of the hydrohalide, use is made of, for example, anilines, pyridines, quinolines, phenylenediamines, α-and β-naphthylamines or imidazoles. The amine hydrohalides are used as an aqueous solution, and water is used in a from 1.9-to 11.9-fold molar excess over the amine (hydrochloride).

EP-A 0 789 013 relates to a process for preparing alcohol chlorides having from 6 to 16 carbon atoms by reacting the corresponding alcohols with hydrogen chloride in the presence of an aqueous alkylpyridine hydrochloride solution which is not specified further, with the reaction being carried out at a temperature below the boiling point of the alkyl chloride and the alkyl chloride formed being distilled off overhead with the aid of additional concentrated hydrochloric acid fed in.

A process for preparing tertiary alkyl chlorides from the corresponding alcohols is described in U.S. Pat. No. 3,852,368. The reaction is carried out in the presence of an organic solvent such as heptane or benzene and of the aqueous solution of an amine. Amines used are tributylamine, triethylamine, n-butylamine or pyridine and water is used in an at least 1.4-fold molar excess over the amine.

DE-A 199 26 165 relates to a process for preparing 1,3-dichloropropane by reacting bis(3-hydroxypropyl)ether with hydrogen chloride in the presence of tertiary basic nitrogen compounds or other tertiary aliphatic bases as catalysts. Suitable tertiary basic nitrogen compounds are pyridine, alkylpyridine, quinoline or trialkylamine, with the tertiary basic nitrogen compounds being present in admixture with water and the ratio of base to water being 0.87-1.18:1 (molar).

DE-A 214 98 22 relates to a continuous process for preparing hydrocarbons which are chlorinated in the 1,4 or 1,5 positions by reaction of a liquid reaction mixture comprising 1,4- or 1,5-diols and/or corresponding cyclic ethers and hydrogen chloride. The reaction is carried out in the presence of a catalyst (tributylamine hydrochloride or N,N-dimethylamine hydrochloride) and water, with the amount of water used being not less than 31 mol %, based on the amount of catalyst.

R. X. Ren et al., Organic Letters, Volume 3 (2001), 3727-3728, describes the reaction of alcohols in the presence of ionic liquids (1-n-butyl-3-methylimidazolium halides) and Brönsted acids at room temperature. In the case of the Brönsted acid HCl used in aqueous form, it is stated that no reaction with n-butyl alcohol occurs in the presence of the ionic liquid (as chloride) over a period of >48 hours.

The literature also describes processes for preparing haloalkanes in which the corresponding alcohol is reaction with hydrogen halide without addition of additional water.

JP-A 2002179600 describes a process for preparing high-purity 3-chloro-1-propanol from 1,3-propanediol and gaseous hydrogen chloride in the presence of a catalyst. Catalysts used are, in particular, zeolites, but quaternary ammonium salts such as tetrabutylammonium and benzyltrimethylammonium chloride, ammonium salts having octyl or octadecyl radicals and phosphonium salts can also be used. To be able to carry out the reaction selectively, the temperature must not exceed 100° C.

JP-A 2001288127 relates to a process for preparing alkyl chlorides from alcohol and gaseous hydrogen chloride in the presence of C₆-C₂₀-alkyldimethylamines as catalyst. The reaction is carried out at 130° C., and the catalyst content is 20 mol % based on the amount of alcohol used. In a comparative experiment, octyltrimethylammonium chloride is used as catalyst. However, the use of this catalyst is described as unfavorable in JP-A 2001288127 because the reaction proceeds more slowly and equimolar amounts of catalyst relative to the alcohol used are necessary. As a consequence, the use of octyltrimethylammonium chloride does not form a key part of the disclosure of JP-A 2001288127 because its use is advised against.

It is an object of the present invention to provide a process for preparing haloalkanes which is improved over the processes known from the prior art. Improvements should be achieved in the space-time yield, the yield and purity of the product and/or the conversion.

We have found this object is achieved by a process in which the halogenation of alcohol with hydrogen halide is carried out so that the reaction of the alcohol with the hydrogen halide occurs in the presence of an ionic liquid at a temperature which is above 100° C. for at least part of the time and, at least at the time of commencement of the reaction, the water content is not more than 25 mol % based on the amount of ionic liquid. For the purposes of the present invention, octyltrimethylammonium chloride is excluded from the ionic liquid.

Compared to processes which employ aqueous solutions of ionic liquids or of the bases corresponding to the ionic liquids, the process of the present invention has the advantage that an improvement in the space-time yield, i.e. accelerated reaction of alcohols with hydrogen halides, can be achieved thereby. Furthermore, the process of the invention is found to give an improved yield compared to the process using ionic liquids in aqueous solution. The process of the present invention also has advantages over the previously known processes which are carried out without addition of additional water. The use of ionic liquids in place of the corresponding bases has the advantage that the product (haloalkane) can be prepared with high selectivity and, in addition, an increase in the reaction temperature gives a significantly improved conversion.

As a result of the optimization of the process for preparing haloalkanes, i.e. the use of ionic liquids at an elevated reaction temperature and, at least initially, the absence of water or the substantial absence of water, a significant shortening of the reaction time of the alcohols with hydrogen halide, and increased conversion and an improved product purity can be achieved, which leads to cost minimization, in particular in respect of relatively large or industrial batches.

A further advantage of the process of the present invention becomes apparent in embodiments in which the water liberated in the reaction is removed continuously from the system in that hydrogen halides, in particular HCl, is less corrosive in low-water systems, so that the apparatus, especially the reaction vessel, is protected. This enables the effort and costs required for maintenance and repair of the plants to be reduced.

Ionic liquids, also referred to as liquid salts, are in general terms salt melts whose melting point is usually below. 100° C. (Ionic Liquids in Synthesis by P. Wasserscheid and T. Welton (Editors), 2003, pp. 41-43, Wiley-VCH Verlag, Weinheim (Germany)). It should be pointed out that the maximum melting point of 100° C. is an arbitrary limit, since compounds which have a melting point higher than 100° C. and can nevertheless be used as ionic liquids are also known.

For the purposes of the process of the present invention, ionic liquids are compounds which have at least one positive charge and at least one negative charge but are overall electrically neutral and have a melting point below 200° C., preferably below 150° C., particularly preferably below 100° C. For the purposes of the present invention, octyltrimethylammonium chloride is excluded from the ionic liquid.

The ionic liquids can also have a plurality of positive or negative charges, for example from 1 to 5, preferably from 1 to 4, particularly preferably from 1 to 3, very particularly preferably 1 or 2, but in particular 1 positive charge and 1 negative charge.

The charges can also be located in various localized or delocalized regions within a molecule, i.e. in a betaine-like fashion, or can each be present on a separate anion and cation. Preference is given to ionic liquids which are made up of at least one cation and at least one anion. Cation and anion can, as indicated above, be singly or multiply charged, preferably singly charged. As anion and as cation of an ionic liquid, all anions and cations are conceivable in principle.

Of course, mixtures of various ionic liquids or mixtures of ionic liquids with metal salts such as AlCl₃, FeCl₃, ZnCl₂ or CoCl₃ are also conveivable.

Preferred ionic liquids have a molecular weight of less than 1000 g/mol, particularly preferably less than 350 g/mol.

Preferred ionic liquids have one each of the anions and cations listed below. All combinations of anions and cations are encompassed, including those in which anion and cation are of different weighting, e.g. the combination of a still more preferred cation with a more preferred anion.

All cations are in principle conceivable as cations.

Preference is given to cations selected from among the compounds of the formulae (a) to (w), 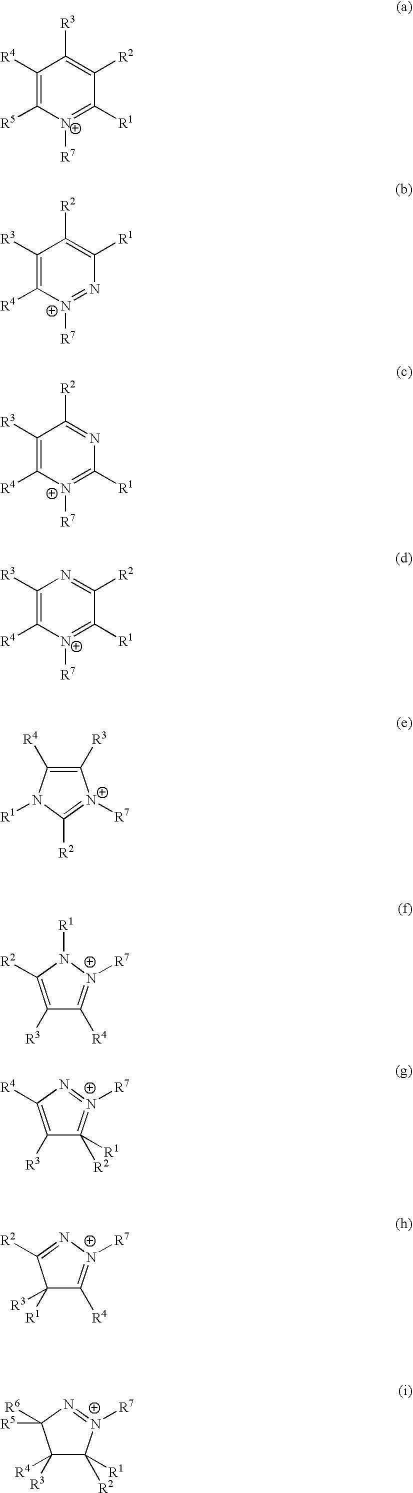 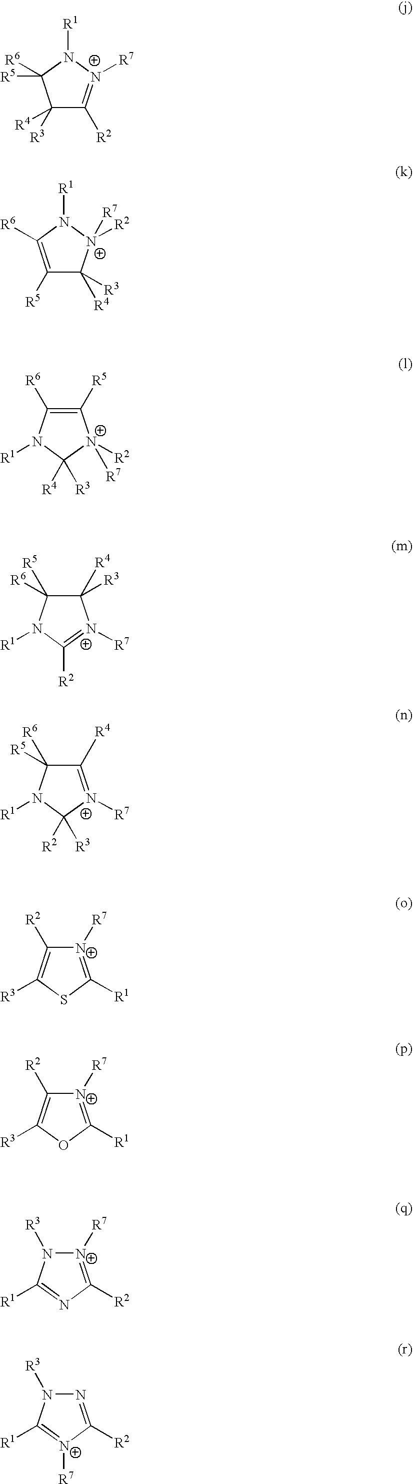 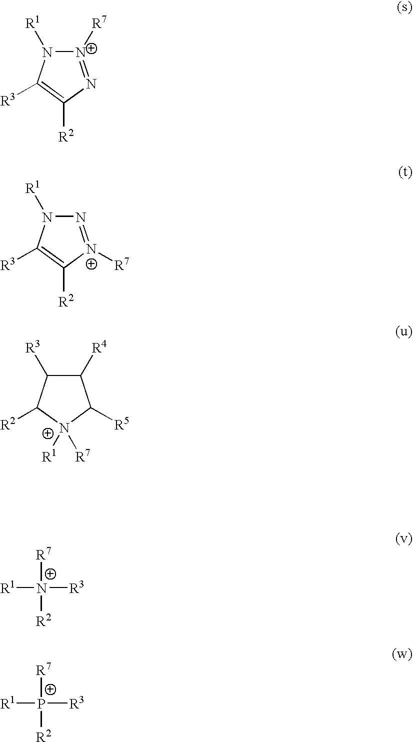
and also oligomers and polymers in which these structures are present,
where
R¹, R², R³, R⁴, R⁵, R⁶ and R⁷ are each, independently of one another, hydrogen, C₁-C₁₈-alkyl, C₁-C₁₈-alkyl which may be interrupted by one or more oxygen and/or sulfur atoms and/or one or more substituted or unsubstituted imino groups, C₆-C₁₂-aryl, C₅-C₁₂-cycloalkyl or a five- or six-membered, oxygen-, nitrogen- and/or sulfur-containing heterocycle or two of them together form an unsaturated, saturated or aromatic ring which may be interrupted by one or more oxygen and/or sulfur atoms and/or one or more substituted or unsubstituted imino groups, where the radicals mentioned may each be substituted by functional groups, aryl, alkyl, aryloxy, alkyloxy, halogen and/or heterocycles. The radicals mentioned can, if desired, independently bear one or more of these substituents. In the case of the ammonium ions (v), R¹, R², R³ and R⁷ are not all an unsubstituted alkyl radical.
R⁷ can also be C₁-C₁₈-alkyloyl (alkylcarbonyl), C₁-C₁₈-alkyloxycarbonyl, C₅-C₁₂-cycloalkylcarbonyl or C₆-C₁₂-aryloyl (arylcarbonyl), where the radicals mentioned may each be substituted by functional groups, aryl, alkyl, aryloxy, alkyloxy, halogen and/or heterocycles. The radicals mentioned can, if desired, independently bear one or more of these substituents.

If one of more of the radicals R¹-R⁷ have an alkyl fragment, this is in each case preferably a C₁-C₈-alkyl fragment which may be either unsubstituted or bear one or more of the above substituents. Here, the radicals, R¹-R⁷ can have identical or different alkyl fragments, in particular identical or different C₁-C₈-alkyl fragments.

If the abovementioned cations (a) to (w) have one or more (further) free electron pairs, for example in the heterocycle and/or the radicals R¹-R⁷, then the forms of these cations in which one or more of these free electron pairs of the cations have been additionally protonated, for example by reaction with hydrogen halide, are also encompassed by the present invention.

Definitions in these Formulae:

Alkyl: C₁-₁₈-alkyl which may be substituted by functional groups, aryl, alkyl, aryloxy, alkyloxy, halogen and/or heterocycles or be unsubstituted, for example methyl, ethyl, propyl, isopropyl, n-butyl, sec-butyl, tert-butyl, pentyl, hexyl, heptyl, octyl, 2-ethylhexyl, 2,4,4-trimethylpentyl, decyl, dodecyl, tetradecyl, heptadecyl, octadecyl, 1,1-dimethylpropyl, 1,1-dimethylbutyl, 1,1,3,3-tetramethylbutyl, benzyl, 1-phenylethyl, 2-phenylethyl, α,α-dimethylbenzyl, benzhydryl, p-tolylmethyl, 1-(p-butylphenyl)ethyl, p-chlorobenzyl, 2,4-dichlorobenzyl, p-methoxybenzyl, m-ethoxybenzyl, 2-cyanoethyl, 2-cyanopropyl, 2-methoxycarbonylethyl, 2-ethoxycarbonylethyl, 2-butoxycarbonylpropyl, 1,2-di(methoxycarbonyl)ethyl, 2-methoxyethyl, 2-ethoxyethyl, 2-butoxyethyl, diethoxymethyl, diethoxyethyl, 1,3-dioxolan-2-yl, 1,3-dioxan-2-yl, 2-methyl-1,3-dioxolan-2-yl, 4-methyl-1,3-dioxolan-2-yl, 2-isopropoxyethyl, 2-butoxypropyl, 2-octyloxyethyl, chloromethyl, 2-chloroethyl, trichloromethyl, trifluoromethyl, 1,1-dimethyl-2-chloroethyl, 2-methoxyisopropyl, 2-ethoxyethyl, 2,2,2-trifluoroethyl, 2-hydroxyethyl, 2-hydroxypropyl, 3-hydroxypropyl, 4-hydroxybutyl, 6-hydroxyhexyl, 2-aminoethyl, 2-aminopropyl, 3-aminopropyl, 4-aminobutyl, 6-aminohexyl, 2-methylaminoethyl, 2-methylaminopropyl, 3-methylaminopropyl,. 4-methylaminobutyl, 6-methylaminohexyl, 2-dimethylaminoethyl, 2-dimethylaminopropyl, 3-dimethylaminopropyl, 4-dimethylaminobutyl, 6-dimethylaminohexyl, 2-hydroxy-2,2-dimethylethyl, 2-phenoxyethyl, 2-phenoxypropyl, 3-phenoxypropyl, 4-phenoxybutyl, 6-phenoxyhexyl, 2-methoxyethyl, 2-methoxypropyl, 3-methoxypropyl, 4-methoxybutyl, 6-methoxyhexyl, 2-ethoxyethyl, 2-ethoxypropyl, 3-ethoxypropyl, 4-ethoxybutyl or 6-ethoxyhexyl, and
C₁-C₁₈-alkyl which may be interrupted by one or more oxygen and/or sulfur atoms and/or one or more substituted or unsubstituted imino groups or be uninterrupted, for example 5-hydroxy-3-oxapentyl, 8-hydroxy-3,6-dioxaoctyl, 11-hydroxy-3,6,9-trioxaundecyl, 7-hydroxy-4-oxaheptyl, 11-hydroxy-4,8-dioxaundecyl, 15-hydroxy-4,8,12-trioxapentadecyl, 9-hydroxy-5-oxanonyl, 14-hydroxy-5,10-oxatetradecyl, 5-methoxy-3-oxapentyl, 8-methoxy-3,6-dioxaoctyl, 11-methoxy-3,6,9-trioxaundecyl, 7-methoxy-4-oxaheptyl, 11-methoxy-4,8-dioxaundecyl, 15-methoxy-4,8,12-trioxapentadecyl, 9-methoxy-5-oxanonyl, 14-methoxy-5,10-oxatetradecyl, 5-ethoxy-3-oxapentyl, 8-ethoxy-3,6-dioxaoctyl, 11-ethoxy-3,6,9-trioxaundecyl, 7-ethoxy-4-oxaheptyl, 11-ethoxy-4,8-dioxaundecyl, 15-ethoxy-4,8,12-trioxapentadecyl, 9-ethoxy-5-oxanonyl, 14-ethoxy-5,10-oxatetradecyl, butylthiomethyl, 2-dodecylthioethyl or 2-phenylthioethyl.

If two radicals form a ring, these radicals can together form 1,3-propylene, 1,4-butylene, 2-oxa-1,3-propylene, 1-oxa-1,3-propylene, 2-oxa-1,3-propylene, 1-oxa-1,3-propenylene, 1-aza-1,3-propenylene, 1-C₁-C₄-alkyl-1-aza-1,3-propenylene, 1,4-buta-1,3-dienylene, 1-aza-1,4-buta-1,3-dienylene or 2-aza-1,4-buta-1,3-dienylene.

The number of oxygen and/or sulfur atoms and/or imino groups is not restricted. In general, there are not more than 5 such atoms/groups in the radical, preferably not more than 4 and very particularly preferably not more than 3.

Furthermore, there is generally at least one carbon atom, preferably at least two carbon atoms, present between two heteroatoms (S, N, O).

Substituted and unsubstituted imino groups can be, for example, imino, methylimino, isopropylimino, n-butylimino or tert-butylimino.

Further Meanings:

Functional groups: carboxyl, carboxamide, hydroxyl, amino, C₁-C₄-alkylamino, Di(C₁-C₄-alkyl)amino, C₁-C₄-alkyloxycarbonyl, cyano or C₁-C₄-alkyloxy,
Aryl: C₆-C₁₂-aryl which may be substituted by functional groups, aryl, alkyl, aryloxy, alkyloxy, halogen and/or heterocycles or be unsubstituted, for example phenyl, tolyl, xylyl, α-naphthyl, β-naphthyl, 4-diphenylyl, chlorophenyl, dichlorophenyl, trichlorophenyl, difluorophenyl, methylphenyl, dimethylphenyl, trimethylphenyl, ethylphenyl, diethylphenyl, isopropylphenyl, tert-butylphenyl, dodecylphenyl, methoxyphenyl, dimethoxyphenyl, ethoxyphenyl, hexyloxyphenyl, methylnaphthyl, isopropylnaphthyl, chloronaphthyl, ethoxynaphthyl, 2,6-dimethylphenyl, 2,4,6-trimethylphenyl, 2,6-diethoxyphenyl, 2,6-dichlorophenyl, 4-bromophenyl, 2- or 4-nitrophenyl, 2,4- or 2,6-dinitrophenyl, 4-dimethylaminophenyl, 4-acetylphenyl, methoxyethylphenyl or ethoxyethylphenyl,
Cycloalkyl: C₅-C₁₂-cycloalkyl which may be substituted by functional groups, aryl, alkyl, aryloxy, alkyloxy, halogen and/or heterocycles or be unsubstituted, for example cyclopentyl, cyclohexyl, cyclooctyl, cyclododecyl, methylcyclopentyl, dimethylcyclopentyl, methylcyclohexyl, dimethylcyclohexyl, diethylcyclohexyl, butylcyclohexyl, methoxycyclohexyl, dimethoxycyclohexyl, diethoxycyclohexyl, butylthiocyclohexyl, chlorocyclohexyl, dichlorocyclohexyl, dichlorocyclopentyl or a saturated or unsaturated bicyclic system such as norbomyl or norbomenyl,
Heterocycle: a 5- or 6-membered, oxygen-, nitrogen- and/or sulfur-containing heterocycle, for example furyl, thienyl, pyrryl, pyridyl, indolyl, benzoxazolyl, dioxolyl, dioxyl, benzimidazolyl, benzthiazolyl, dimethylpyridyl, methylquinolyl, dimethylpyrryl, methoxyfuryl, dimethoxypyridyl, difluoropyridyl, methylthienyl, isopropyithienyl or tert-butylthienyl, with the heterocycle being able to be substituted by functional groups, aryl, alkyl, aryloxy, alkyloxy, halogen and/or further heterocycles or be unsubstituted, and
C₁-C₄-Alkyl: for example methyl, ethyl, propyl, isopropyl, n-butyl, sec-butyl or tert-butyl.
C₁-C₁₈-Alkyloyl (alkylcarbonyl) can be, for example, acetyl, propionyl, n-butyloyl, sec-butyloyl, tert-butyloyl, 2-ethylhexylcarbonyl, decanoyl, dodecanoyl, chloroacetyl, trichloroacetyl or trifluoroacetyl.
C₁-C₁₈-Alkyloxycarbonyl can be, for example, methyloxycarbonyl, ethyloxycarbonyl, propyloxycarbonyl, isopropyloxycarbonyl, n-butyloxycarbonyl, sec-butyloxycarbonyl, tert-butyloxycarbonyl, hexyloxycarbonyl, 2-ethylhexyloxycarbonyl or benzyloxycarbonyl.
C₁-C₁₂-Cycloalkylcarbonyl can be, for example cyclopentylcarbonyl, cyclohexylcarbonyl or cyclododecylcarbonyl.
C₆-C₁₂-Aryloyl (arylcarbonyl) can be, for example, benzoyl, toluyl, xyloyl, α-naphthoyl, β-naphthoyl, chlorobenzoyl, dichlorobenzoyl, trichlorobenzoyl or trimethylbenzoyl.

In the case of alkyloxy (alkoxy) and aryloxy substituents, their alkyl or aryl fragments have the above definitions for alkyl or aryl.
R¹, R², R³, R⁴, R⁵ and R⁶ are preferably each, independently of one another, hydrogen, methyl, ethyl, n-butyl, 2-hydroxyethyl, 2-cyanoethyl, 2-(methoxycarbonyl)ethyl, 2-(ethoxycarbonyl)ethyl, 2-(n-butoxycarbonyl)ethyl, dimethylamino, diethylamino or chlorine.
R⁷ is preferably hydrogen, methyl, ethyl, n-butyl, 2-hydroxyethyl, 2-cyanoethyl, 2-(methoxycarbonyl)ethyl, 2-(ethoxycarbonyl)ethyl, 2-(n-butoxycarbonyl)ethyl, acetyl, propionyl, t-butyryl, methoxycarbonyl, ethoxycarbonyl or n-butoxycarbonyl.

More preferred pyridinium ions (a) are those in which at least one of the radicals R¹to R⁵ is methyl, ethyl or chlorine, R⁷ is hydrogen, acetyl, methyl, ethyl or n-butyl and all others are hydrogen, or R³ is dimethylamino, R⁷ is hydrogen, acetyl, methyl, ethyl or n-butyl and all others are hydrogen or R⁷ is hydrogen, acetyl, methyl, ethyl or n-butyl and all others are hydrogen or R² is carboxyl or carboxamide, R⁷ is hydrogen, acetyl, methyl, ethyl or n-butyl and all others are hydrogen or R¹ and R² or R² and R³ are each 1,4-buta-1,3-dienylene, R⁷ is hydrogen, acetyl, methyl, ethyl or n-butyl and all others are hydrogen.

More preferred pyridazinium ions (b) are those in which one of the radicals R¹ to R⁴ is methyl or ethyl, R⁷ is hydrogen, acetyl, methyl, ethyl or n-butyl and all others are hydrogen or R⁷ is hydrogen, acetyl, methyl, ethyl or n-butyl, and all others are hydrogen

More preferred pyrimidinium ions (c) are those in which R² to R⁴ are each hydrogen or methyl, R⁷ is hydrogen, acetyl, methyl, ethyl or n-butyl and R¹ is hydrogen, methyl or ethyl, or R² and R⁴ are each methyl, R³ is hydrogen and R¹ is hydrogen, methyl or ethyl and R⁷ is hydrogen, acetyl, methyl, ethyl or n-butyl.

More preferred pyrazinium ions (d) are those in which
R¹ to R⁴ are all methyl and
R⁷ is hydrogen, acetyl, methyl, ethyl or n-butyl or R⁷ is hydrogen, acetyl, methyl, ethyl or n-butyl and all others are hydrogen.

More preferred imidazolium ions (e) are those in which, independently of one another,
R¹ is selected from among methyl, ethyl, n-propyl, n-butyl, n-pentyl, n-octyl, n-decyl, n-dodecyl, 2-hydroxyethyl and 2-cyanoethyl,
R⁷ is hydrogen, acetyl, methyl, ethyl, n-propyl or n-butyl and
R² to R⁴ are each, independently of one another, hydrogen, methyl or ethyl.

More preferred 1H-pyrazolium ions (f) are those in which, independently of one another,
R¹ is selected from among hydrogen, methyl and ethyl,
R², R³ and R⁴ are selected from among hydrogen and methyl and
R⁷ is selected from among hydrogen, acetyl, methyl, ethyl and n-butyl.

More preferred 3H-pyrazolium ions (g) are those in which, independently of one another,
R¹ is selected from among hydrogen, methyl and ethyl,
R², R³ and R⁴ are selected from among hydrogen and methyl and
R⁷ is selected from among hydrogen, acetyl, methyl, ethyl and n-butyl.

More preferred 4H-pyrazolium ions (h) are those in which, independently of one another,
R¹ to R⁴ are selected from among hydrogen and methyl and
R⁷ is selected from among hydrogen, acetyl, methyl, ethyl and n-butyl.

More preferred 1-pyrazolinium ions (i) are those in which, independently of one another,
R¹ to R⁶ are selected from among hydrogen and methyl and
R⁷ is selected from among hydrogen, acetyl, methyl, ethyl and n-butyl.

More preferred 2-pyrazolinium ions (j) are those in which, independently of one another,
R¹ is selected from among hydrogen, methyl, ethyl and phenyl,
R⁷ is selected from among hydrogen, acetyl, methyl, ethyl and n-butyl and
R² to R⁶ are selected from among hydrogen and methyl.

More preferred 3-pyrazolinium ions (k) are those in which, independently of one another,
R¹ and R² are selected from among hydrogen, methyl, ethyl and phenyl,
R⁷ is selected from among hydrogen, acetyl, methyl, ethyl and n-butyl and
R³ to R⁶ are selected from among hydrogen and methyl.

More preferred imidazolinium ions (I) are those in which, independently of one another,
R¹ and R² are selected from among hydrogen, methyl, ethyl, n-butyl and phenyl,
R⁷ is selected from among hydrogen, acetyl, methyl, ethyl and n-butyl and
R³ and R⁴ are selected from among hydrogen, methyl and ethyl and
R⁵ and R⁶ are selected from among hydrogen and methyl.

More preferred imidazolinium ions (m) are those in which, independently of one another,
R¹ and R² are selected from among hydrogen, methyl and ethyl,
R⁷ is selected from among hydrogen, acetyl, methyl, ethyl and n-butyl and
R³ to R⁶ are selected from among hydrogen and methyl.

More preferred imidazolinium ions (n) are those in which, independently of one another,
R¹, R² and R³ are selected from among hydrogen, methyl and ethyl,
R⁷ is hydrogen, acetyl, methyl, ethyl and n-butyl and
R⁴ to R⁶ are selected from among hydrogen and methyl.

More preferred thiazolium ions (o) or oxazolium ions (p) are those in which, independently of one another,
R¹ is selected from among hydrogen, methyl, ethyl and phenyl,
R⁷ is selected from among hydrogen, acetyl, methyl, ethyl and n-butyl and
R² and R³ are selected from among hydrogen and methyl.

More preferred 1,2,4-triazolium ions (q) and (r) are those in which, independently of one another,
R¹ and R² are selected from among hydrogen, methyl, ethyl and phenyl,
R⁷ is selected from among hydrogen, acetyl, methyl, ethyl and n-butyl and
R³ is selected from among hydrogen, methyl and phenyl.

More preferred 1,2,3-triazolium ions (s) and (t) are those in which, independently of one another,
R¹ is selected from among hydrogen, methyl and ethyl,
R⁷ is selected from among hydrogen, acetyl, methyl, ethyl and n-butyl and
R² and R³ are selected from among hydrogen and methyl or
R² and R³ form a 1,4-buta-1,3-dienylene group and all others are hydrogen.

More preferred pyrrolidinium ions (u) are those in which, independently of one another
R¹ and R⁷ are selected from among hydrogen, acetyl, methyl, ethyl and n-butyl and
R², R³, R⁴ and R⁵ are each hydrogen.

More preferred ammonium ions (v) are those in which, independently of one another,
R⁷ is selected from among hydrogen and acetyl and
R¹, R² and R³ are selected from among 2-hydroxyethyl, benzyl and phenyl.

More preferred phosphonium ions (w) are those in which, independently of one another,
R⁷ is selected from among hydrogen, acetyl, methyl, ethyl and n-butyl and
R¹, R² and R³ are selected from among phenyl, phenoxy, ethoxy and n-butoxy.

Among the abovementioned more preferred cations (a-w), the respective phosphonium (w), pyridinium (a) and imidazolium ions (e) are even more preferred, especially the respective pyridinium (a) and imidiazolium ion (e).
Particularly preferred cations are 1-methylimidazolium, 1-ethylimidazolium, 1-propylimidazolium, 1-butylimidazolium, 2-ethylpyridinium, 1-ethyl-3-methylimidazolium, 1-n-butyl-3-ethylimidazolium, 1,2-dimethylpyridinium, 1-methyl-2-ethylpyridinium, 1-methyl-2-ethyl-6-methylpyridinium, N-methylpyridinium, 1-butyl-2-methylpyridinium, 1-butyl-2-ethylpyridinium, 1-butyl-2-ethyl-6-methylpyridinium, N-butylpyridinium, 1-butyl-4-methylpyridinium, 1,3-dimethylimidazolium, 1,2,3-trimethylimidazolium, 1-n-butyl-3-methylimidazolium, 1,3,4,5-tetramethylimidazolium, 1,3,4-trimethylimidazolium, 1,2-dimethylimidazolium, 1-butyl-2,3-dimethylimidazolium, 3,4-dimethylimidazolium, 2-ethyl-3,4-dimethylimidazolium, 3-methyl-2-ethylimidazolium, 3-butyl-1-methylimidazolium, 3-butyl-1-ethylimidazolium, 3-butyl-1,2-dimethylimidazolium, 1,3-di-n-butylimidazolium, 3-butyl-1,4,5-trimethylimidazolium, 3-butyl-1,4-dimethylimidazolium, 3-butyl-2-methylimidazolium, 1,3-dibutyl-2-methylmidazolium, 3-butyl-4-methylimidazolium, 3-butyl-2-ethyl-4-methylimidazolium 3-butyl-2-ethylimidazolium, 1-methyl-3-octylimidazolium and 1-decyl-3-methylimidazolium.
Very particularly preferred cations are 1-methylimidazolium, 1-ethylimidazolium, 1-propylimidazolium, 1-butylimidazolium, 2-ethylpyridinium, 1-ethyl-3-methylimidazolium, 1-butyl-4-methylpyridinium, 1-n-butyl-3-methylimidazolium and 1-n-butyl-3-ethylimidazolium.

All anions are in principle conceivable as anions.

Preferred anions are halides (F⁻, Cl⁻, Br⁻, I³¹ ), alkylcarboxylate (C₁-C₁₈-alkyl-CO₂⁻), tosylate (p-CH₃C₆H₄SO₃⁻), sulfonate (C₁-C₁₈-alkyl-SO₃⁻), dialkylphosphate (di(C₁-C₁₈-alkyl)-PO₄⁻), bis(trifluoromethylsulfonyl)imide ((CF₃SO₂)₂N⁻), trifluoroacetate (CF₃COO⁻), triflate (CF₃SO₃⁻), sulfate (SO₄²⁻), hydrogensulfate (HSO₄⁻), methylsulfate (CH₃OSO₃⁻), ethylsulfate (C₂H₅OSO₃⁻), sulfite (SO₃²⁻), hydrogensulfite (HSO₃⁻), chloroaluminates (AlCl₄⁻), (Al₂Cl₇⁻), (Al₃Cl₁₀⁻), bromoaluminates (AlBr₄⁻), nitrite (NO₂⁻), nitrate (NO₃⁻), chlorocuprate (CuCl₂⁻), phosphate (PO₄³⁻), hydrogenphosphate (HPO₄²⁻), dihydrogenphosphate (H₂PO₄⁻), carbonate (CO₃²⁻) and hydrogencarbonate (HCO₃⁻).

More preferred anions are halides, acetate, methanesulfonate, tosylate, sulfate, hydrogensulfate, phosphate, hydrogenphosphate, dihydrogenphosphate, dialkylphosphate and bis(trifluoromethylsulfonyl)imide.

Particularly preferred anions are chloride, bromide, hydrogensulfate and diethylphosphate.

Particularly preferred ionic liquids are selected from the group consisting of:
1-methylimidazolium chloride, 1-methylimidazolium bromide, 1-methylimidazolium fluoride, 1-methylimidazolium iodide, 1-methylimidazolium hydrogensulfate, 1-methylimidazolium sulfate, 1-methylimidazolium methanesulfonate, 1-methylimidazolium tosylate, 1-methylimidazolium diethylphosphate, 1-ethylimidazolium chloride, 1-ethylimidazolium bromide, 1-ethylimidazolium fluoride, 1-ethylimidazolium iodide, 1-ethylimidazolium hydrogensulfate, 1-ethylimidazolium sulfate, 1-ethylimidazolium methanesulfate, 1-ethylimidazolium tosylate, 1-ethylimidazolium-diethylphosphate, 1-propylimidazolium chloride, 1-propylimidazolium bromide, 1-propylimidazolium fluoride, 1-propylimidazolium iodide, 1-propylimidazolium hydrogensulfate, 1-propylimidazolium sulfate, 1-propylimidazolium methanesulfate, 1-propylimidazolium tosylate, 1-propylimidazolium diethylphosphate, 1-butylimidazolium chloride, 1-butylimidazolium bromide, 1-butylimidazolium fluoride, 1-butylimidazolium iodide, 1-butylimidazolium hydrogensulfate, 1-butylimidazolium sulfate, 1-butylimidazolium methanesulfonate, 1-butylimidazolium tosylate, 1-butylimidazolium diethylphosphate, 2-ethylpyridinium chloride, 2-ethylpyridinium bromide, 2-ethylpyridinium iodide, 2-ethylpyridinium hydrogensulfate, 2-ethylpyridinium sulfate, 2-ethylpyridinium methanesulfonate, 2-ethylpyridinium tosylate, 2-ethylpyridinium diethylphosphate, 1-ethyl-3-methylimidazolium chloride, 1-ethyl-3-methylimidazolium bromide, 1-ethyl-3-methylimidazolium fluoride, 1-ethyl-3-methylimidazolium iodide, 1-ethyl-3-methylimidazolium hydrogensulfate, 1-ethyl-3-methylimidazolium sulfate, 1-ethyl-3-methylimidazolium methanesulfonate, 1-ethyl-3-methylimidazolium tosylate, 1-ethyl-3-methylimidazolium diethylphosphate, 1-n-butyl-3-methylimidazolium chloride, 1-n-butyl-3-methylimidazolium bromide, 1-n-butyl-3-methylimidazolium fluoride, 1-n-butyl-3-methylimidazolium iodide, 1-n-butyl-3-methylimidazolium hydrogensulfonate, 1-n-butyl-3-methylimidazolium sulfate, 1-n-butyl-3-methylimidazolium methanesulfonate, 1-n-butyl-3-methylimidazolium tosylate, 1-n-butyl-3-methylimidazolium diethylphosphate, 1-n-butyl-3-ethylimidazolium chloride, 1-n-butyl-3-ethylimidazolium bromide, 1-n-butyl-3-ethylimidazolium fluoride, 1-n-butyl-3-ethylimidazolium iodide, 1-n-butyl-3-ethylimidazolium hydrogensulfate, 1-n-butyl-3-ethylimidazolium sulfate, 1-n-butyl-3-ethylimidazolium methanesulfonate, 1-n-butyl-3-ethylimidazolium tosylate and 1-n-butyl-3-ethylimidazolium diethylphosphate.

Very particularly preferred ionic liquids are selected from the group consisting of:
1-methylimidazolium chloride, 1-methylimidazolium bromide, 1-methylimidazolium hydrogensulfate, 2-ethylpyridinium chloride, 2-ethylpyridinium bromide, 2-ethylpyridinium hydrogensulfate, 1-ethyl-3-methylimidazolium chloride, 1-ethyl-3-methylimidazolium bromide and 1-ethyl-3-methylimidazolium hydrogensulfate.

In the process of the present invention, it is in principle possible to use all alcohols, including those which have two or more OH groups. If desired, the alcohols can also be monosubstituted or polysubstituted.

Preferred alcohols are: linear, branched or cyclic C₁-C₂₀-alcohols. Greater preference is given to linear, branched or cyclic C₁-C₁₀-alcohols such as sec-butanol, isobutanol, 2-ethylhexanol, 2-propylheptanol, isononanol, cyclohexanol, cyclopentanol, glycol, 1,3-propanediol, 1,4-butanediol, 1,5-pentanediol, 1,6-hexanediol, neopentylglycol, trimethylolpropane, pentaerythritol, glycerol, trimethylolethane, 1,2-propanediol, 1,2-butanediol, 2,3-butanediol, allyl alcohol, propargyl alcohol, diethylene glycol and triethylene glycol.

Particularly preferred alcohols are: 1,6-hexanediol, 1,5-pentanediol, 1,4-butanediol, 1,3-propanediol, glycol, allyl alcohol and propargyl alcohol.

The halogenation in the process of the present invention is carried out using hydrogen halide which is exclusively in gaseous form. Suitable hydrogen halides are hydrogen fluoride, hydrogen chloride, hydrogen bromide and hydrogen iodide, preferably hydrogen chloride and hydrogen bromide, particularly preferably hydrogen chloride.

The process of the present invention is usually carried out so that one of the abovementioned alcohols reacts with one of the abovementioned hydrogen halides. However it is also possible, if desired, to use mixtures of alcohols and/or hydrogen halides.

In the case of alcohols having more than one OH group per molecule, the reaction of the alcohol with hydrogen halide either results in replacement of all OH groups by a halogen or the reaction proceeds so that only part of the OH groups of the corresponding alcohol (per molecule), for example in the case of glycerol or 1,3-propanediol, are replaced by halogens. Consequently, in the case of 1,3-propanediol, either 3-chloropropanol or 1,3-dichloropropane can be prepared in the process of the present invention, with the reaction being controlled via the amount of hydrogen halide added. Preference is given to all OH groups of an alcohol being replaced by a halogen in the process of the present invention.

In the process of the present invention, the ionic liquid is initially placed in a reaction vessel and then brought to a temperature which is above the melting point of the ionic liquid. Hydrogen halide can subsequently be passed into the ionic liquid until saturation of the liquid is reached. The alcohol is then added and subsequent to the addition of alcohol, hydrogen halide is (again) passed in. If appropriate, it is also possible to place the alcohol in the reaction vessel first and to pass the saturated ionic liquid into the alcohol at temperatures which are above the melting point of the ionic liquid. Preference is given to passing the alcohol into the saturated ionic liquid.

The process of the present invention is preferably carried out so that the reaction occurs in the presence of from 0.3 to 3 mol, more preferably from 1 to 3 mol, particularly preferably from 1 to 20 mol, of ionic liquid per mole of OH group to be reacted in the alcohol. If desired, the reaction can also be carried out with a larger molar excess of ionic liquid, but in this case a reduction in the space-time yield is observed as a result of the volume increase. Likewise, an amount of ionic liquid less than 0.3 times the molar amount of OH group is also conceivable.

The process of the present invention is carried out at above 100° C. for at least part of the time. The reaction is preferably carried out at from 110° C. to 150° C. for at least part of the time, more preferably from 120° C. to 145° C. for at least part of the time, particularly preferably from 125° C. to 140° C. for at least part of the time.

In the process of the present invention, the alcohol can be added to the ionic liquid at temperatures below 100° C. The subsequent introduction of the gaseous hydrogen halide into the mixture comprising the alcohol and the ionic liquid can likewise, partly or completely, be carried out at temperatures below 100° C. If the addition of the alcohol to the ionic liquid and/or the subsequent introduction of the gaseous hydrogen halide are/is carried out, partly or completely, at temperatures below 100° C., this preferably occurs at at least 20° C., more preferably at least 50° C., even more preferably at least 75° C. and particularly preferably 85° C. However, a prerequisite for a selective conversion of the alcohol into the corresponding haloalkane is that the introduction of the gaseous hydrogen halide into the mixture comprising the alcohol and the ionic liquid is commenced before the resulting mixture comprising hydrogen halide, the alcohol and the ionic liquid is heated to temperatures above 100° C. for part of the time but this heating procedure is carried out not later than after completion of the introduction of the gaseous hydrogen halide. Only this increase in the reaction temperature to temperatures above 100° C. brings about complete and selective conversion into the desired haloalkane.

The reaction temperature has to be increased to temperatures above 100° C. for a sufficient time. The time for which the temperature is increased to above 100° C. should be not less than 1 minute and is preferably more than 1 minute, more preferably more than 5 minutes, even more preferably more than 15 minutes, particularly preferably more than 30 minutes. The temperature is preferably increased to above 100° C. only after the end of the addition of hydrogen halide to the mixture comprising the alcohol. The reaction temperature is preferably increased to above 100° C. according to a temperature ramp, i.e. the reaction temperature is increased continuously to temperatures above 100° C. after at least part of the hydrogen halide has been added and the reaction mixture is maintained at above 100° C. until the reaction is complete.

However, in one embodiment of the present invention, the entire addition of the hydrogen halide to the mixture comprising the alcohol can be carried out at temperatures above 100° C. If appropriate, the addition of the alcohol to the ionic liquid can also be carried out at temperatures above 100° C. With regard to the preferred, (more preferred, etc.) temperature ranges, the same ranges as given for a reaction carried out with the temperature being increased to above 100° C. for only part of the time applied.

The process of the present invention is preferably carried out at temperatures above 100° C. for only part of the time. Even better selectivities to the haloalkane to be prepared compared to the ether formed as by-product and also an improved conversion can be achieved in this way.

The lower the water content at the beginning of the halogenation reaction, the quicker the reaction of alcohols with hydrogen halides. Accordingly, the water content in the process of the present invention is not more than 25 mol % based on the amount of ionic liquid, at least at the time of commencement of the reaction. Preference is given to a maximum water content of 20 mol %, more preferably a maximum of 10 mol %, even more preferably a maximum of 5 mol %. Particular preference is given to the reaction being water-free or substantially water-free at the time of commencement of the reaction. In the following, “substantially free of water” means a water content in the ppm range.

The haloalkanes prepared by the process of the present invention can be isolated from the reaction mixture after the reaction is complete by methods known to those skilled in the art. If a two-phase system is formed in the reaction mixture, the product (haloalkane) can be isolated by simple phase separation, and, if appropriate, further amounts of product present in the other phase of the two-phase system can be isolated from this phase by means of additional extraction steps or distillation; if a single-phase reaction mixture is formed, the product can be isolated from the reaction mixture by extractional distillation. The product is preferably obtained from the reaction mixture by distillation, in particular using a distillation attachment under reduced pressure. If a two-phase mixture is formed in this distillation, the product can be isolated, for example, by separation, extraction or, if appropriate, by means of additional distillation steps.

In a further embodiment, the process of the present invention is carried out so that the water content is not more than 25 mol %, preferably not more than 20 mol %, more preferably not more than 10 mol %, particularly preferably not more than 5 mol %, based on the amount of ionic liquid, over the entire reaction time. This limitation of the maximum water content over the entire reaction time is achieved by the water of reaction liberated in the reaction of the alcohol with hydrogen halide or the water previously present in the system being continuously removed from the system. Water can, for example, be removed from the system by distillation or by use of desiccants or membranes. Preference is given to distilling off the water. The continuous removal of the water of reaction is advantageous since the reaction proceeds more quickly, the less water is present in the system and because an increase in the content of water of reaction results in a steady slowing of the reaction rate and thus a decrease in the space-time yield. The process of the present invention can be carried out either continuously or batchwise. For the purposes of the present invention, a “continuous process” means that not only is the water removed continuously but the starting materials and, if appropriate, the product are continuously introduced or removed.

In the process of the present invention, the ionic liquids can either be used directly or they are prepared immediately before the reaction of the alcohol with hydrogen halide by passing the hydrogen halide into the appropriate base and saturating the base therewith.

The following examples illustrate the invention:

### EXAMPLE 1

51.7 g of hydrogen chloride gas are passed into 82.0 g of 1-methylimidazole at about 93° C. to prepare the hydrochloride which is liquid under these conditions and this is subsequently heated to 135° C. At this temperature, 59.0 g of 1,6-hexanediol are added and 83.3 g of hydrogen chloride are then passed into the reaction mixture, forming a liquid two-phase mixture. The reaction is complete. The upper phase is separated off to give 66.5 g (81.4%) of 1,6-dichlorohexane which has a purity (GC) of 96.2% and additionally contains 3.2% of bis(6-chlorohexyl)ether.

Distillation of the lower phase gives a further 3.3 g (4.1%) of 1,6-dichlorohexane having a purity of 98.2%.

### COMPARATIVE EXAMPLE 1A

At 135° C., 82.0 g of 1-methylimidazole and 59.0 g of 1,6-hexanediol are placed in a reaction vessel while stirring and 85.6 g of hydrogen chloride are subsequently passed in, forming a liquid two-phase mixture. The conversion is 99.9%. The upper phase is separated off to give 67.3 g (74.1%) of 1,6-dichlorohexane which has a purity (GC) of 88.5% and additionally contains 8.2% of bis(6-chlorohexyl)ether.

### COMPARATIVE EXAMPLE 1B

56.7 g of hydrogen chloride gas are passed into 82.0 g 1-methylimidazole at about 86° C. to prepare the hydrochloride which is liquid under these conditions. 59.0 g of 1,6-hexanediol are subsequently added and hydrogen chloride (49.3 g) is subsequently passed in at 80-86° C. Analysis of the single-phase reaction mixture indicates a conversion of 49.7%. The 1,6-dichlorohexane content corresponds to a yield of 8.7%.

It can be seen from example 1 that the reaction of the alcohol with hydrogen halide at >100° C. in the presence of an ionic liquid offers advantages over the prior art (comparative examples 1A, 1B). In comparative example 1A, the corresponding free base is used instead of the ionic liquid. The total yield of reaction product is lower, with a larger amount of ether being formed as by-product. Compared to comparative example 1B, the reaction is carried out at higher temperatures, which has a positive effect both on the conversion and on the yield (higher selectivity) of reaction product (haloalkane).

### EXAMPLES 2-4

1 mol of base or ionic liquid is placed in a stirred HWS reactor. This initial charge is then heated to 135° C. Hydrogen chloride is passed in until saturation is achieved to prepare the corresponding ionic liquid from the base. In the present case, hydrogen chloride is also passed into the ionic liquid to obtain the same initial conditions as when the free base is used, but this is not absolutely necessary. 0.5 mol of 1,6-hexanediol is subsequently added and hydrogen chloride is then passed in, with the absorption of HCl being monitored by means of a gas burette.

TABLE 1

| | | | Duration of HCl absorption | | |
|---|---|---|---|---|---|
| | | Amount | (min:sec) | | |
| Example | Base/ionic liquid | of water | 500 ml | 1000 ml | 1500 ml |
| 2 | 1-methylimidazole | — | 0:28 | 1:01 | 1:54 |
| 3 | 2-ethylpyridine | — | 0:25 | 0:56 | 1:42 |
| 4 | 1-ethyl-3-methyl- | — | 0:19 | 0:40 | 1:06 |
| | imidazolium | | | | |
| | chloride | | | | |

### COMPARATIVE EXAMPLES 2-5

The comparative examples are carried out in an analogous manner to the examples according to the present invention, except that, in addition to the base or ionic liquid, the amount of water indicated in the table (based on the ionic liquid) is initially placed in the reaction vessel and the solution is subsequently saturated with hydrogen chloride.

TABLE 2

| | | Amount | Duration of HCl absorption | | |
|---|---|---|---|---|---|
| Comparative | | of water | (min:sec) | | |
| Example | Base/ionic liquid | [mol] | 500 ml | 1000 ml | 1500 ml |
| C2 | 1- | 0.5 | 1:07 | 2:13 | 4:13 |
| | methylimidazole | | | | |
| C3 | 2-ethylpyridine | 1 | 0:35 | 1:29 | 2:55 |
| C4A | 1-ethyl-3-methyl- | 0.5 | 1:01 | 1:35 | 2:20 |
| | imidazolium | | | | |
| | chloride | | | | |
| C4B | 1-ethyl-3-methyl- | 1 | 1:03 | 2:33 | 4:50 |
| | imidazolium | | | | |
| | chloride | | | | |
| C5 | tri-n-butylamine | 1 | 0:42 | 1:53 | 4:55 |

The duration of HCl absorption is a measure of the reaction rate. It can be seen from the tables above that the reaction occurs significantly more quickly in the process of the present invention, which results in an improvement in the space-time yield. Assuming complete conversion of HCl passed in and that this behaves as an ideal gas, an HCl absorption of about 1500 ml corresponds to an amount of water of reaction liberated of not more than 30 mol %. Furthermore, the experiments show that when ionic liquids are used at relatively high temperatures and in the initial absence of water, no appreciable slowing of the reaction rate is observed despite an increasing amount of water of reaction (up to a maximum of 30 mol %).

### EXAMPLE 5

118.5 g (1.0 mol) of 1-methylimidazole hydrochloride are placed in a reaction vessel as a melt at a temperature of 135° C. and admixed with 59.0 g (0.5 mol) of solid 1,6-hexanediol. 47.3 g (1.29 mol) of hydrogen chloride gas are subsequently passed as a uniform gas stream into the reaction mixture over a period of 35 minutes. The mixture is subsequently distilled via a distillation attachment under reduced pressure until no more product goes over at an internal temperature of 139° C. and 33 mbar. The distillate is obtained as a two-phase mixture. After the aqueous phase has been separated off, 69.7 g (88.8%) of 1,6-dichlorohexane having a purity (GC) of 98.7% are obtained.

### EXAMPLE 6

At a temperature of 85° C., 44.6 g (1.22 mol) of hydrogen chloride gas are passed into 82.0 g (1.0 mol) of 1-methylimidazole to form the hydrochloride which is liquid under these conditions, the reaction mixture is admixed with 18.0 g (1.0 mol) of water and is subsequently heated to 135° C. At this temperature, 59.0 g (0.5 mol) of 1,6-hexanediol are added and 67.7 g (1.85 mol) of hydrogen chloride gas are then passed into the reaction mixture over a period of 4 hours, resulting in formation of a two-phase mixture. The mixture is subsequently distilled via a distillation attachment under reduced pressure until no more product goes over at an internal temperature of 135° C. and 39 mbar. Separation of the two-phase distillate gives 58.1 g (74.0%) of 1,6-dichlorohexane having a purity (GC) of 98.7%.

### EXAMPLE 7

At a temperature of up to 111° C., 82.0 g (1.0 mol) of 1-methylimidazole are converted into 1-methylimidazole hydrochloride by passing in 45.3 g (1.24 mol) of hydrogen chloride gas, and the reaction product is admixed at 98° C. with 59.0 g (0.5 mol) of solid 1,6-hexanediol. 37.8 g (1.04 mol) of hydrogen chloride gas are subsequently passed as a uniform gas stream into the reaction mixture over a period of 2.5 hours. The temperature is regulated to 83-88° C. for the first 130 minutes, and the reaction mixture is then heated to 135° C. over a period of 20 minutes, the introduction of HCl is stopped and the mixture is stirred for another two hours. The reaction mixture is then distilled via a distillation attachment under reduced pressure until no more product goes over at an internal temperature of 140° C. and 24 mbar. The distillate is obtained as a two-phase mixture. After the aqueous phase has been separated off, 72.7 g (95.5%) of 1,6-dichlorohexane having a purity (GC) of 99.1% are obtained.

Comparison of example 7 with example 5 shows that the use of a temperature ramp, i.e. the reaction of alcohol with hydrogen halide in the presence of an ionic liquid is carried out at temperatures above 100° C. for only part of the time, leads to an improved selectivity and an improved conversion. Furthermore, comparison with example 6 shows that the use of ionic liquids in an aqueous phase leads to a significantly reduced conversion to the desired product (here 1,6-dichlorohexane).

We claim:

1. A process for preparing haloalkanes comprising reacting alcohol with hydrogen halide, wherein the reaction of the alcohol with the hydrogen halide occurs in the presence of an ionic liquid at a temperature which is above 100° C. for at least part of the time and, at least at the time of commencement of the reaction, the water content is not more than 25 mol % based on the amount of ionic liquid, where the ionic liquid is not octyltrimethylammonium chloride.

2. A process as claimed in claim 1, wherein part or all of the hydrogen halide is passed into the mixture comprising the alcohol and the ionic liquid at temperatures below 100° C. and the mixture formed by the addition is heated to temperatures above 100° C. for part of the time.

3. A process as claimed in claim 1, wherein the reaction is carried out at from 100° C. to 150° C.

4. A process as claimed in claim 1 wherein HCl or HBr is used as hydrogen halide.

5. A process as claimed in claim 1, wherein an alcohol selected from the group consisting of sec-butanol, isobutanol, 2-ethylhexanol, 2-propylheptanol, isononanol, cyclohexanol, cyclopentanol, glycol, 1,3-propanediol, 1,4-butanediol, 1,5-pentanediol, 1,6-hexanediol, neopentyl glycol, trimethylolpropane, pentaerythritol, glycerol, trimethylolethane, 1,2-propanediol, 1,2-butanediol, 2,3-butanediol, allyl alcohol, propargyl alcohol, diethylene glycol and triethylene glycol, in particular selected from the group consisting of 1,6-hexanediol, 1,5-pentanediol, 1,4-butanediol, 1,3-propanediol, glycol, allyl alcohol and propargyl alcohol, is used.

6. A process as claimed in claim 1, wherein an ionic liquid which comprises one each of the following cations and anions,
where the cation is selected from the group consisting of: 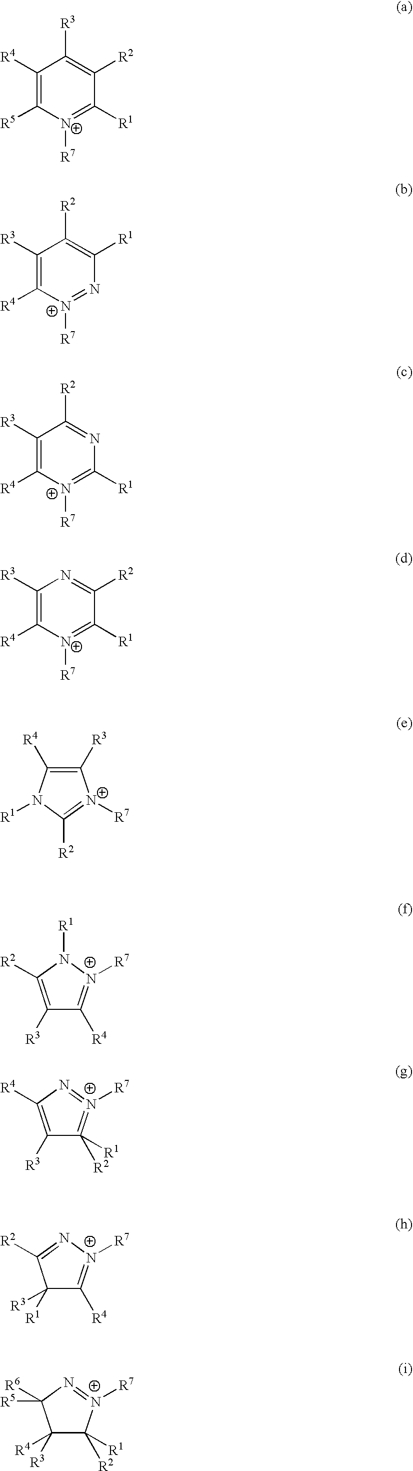 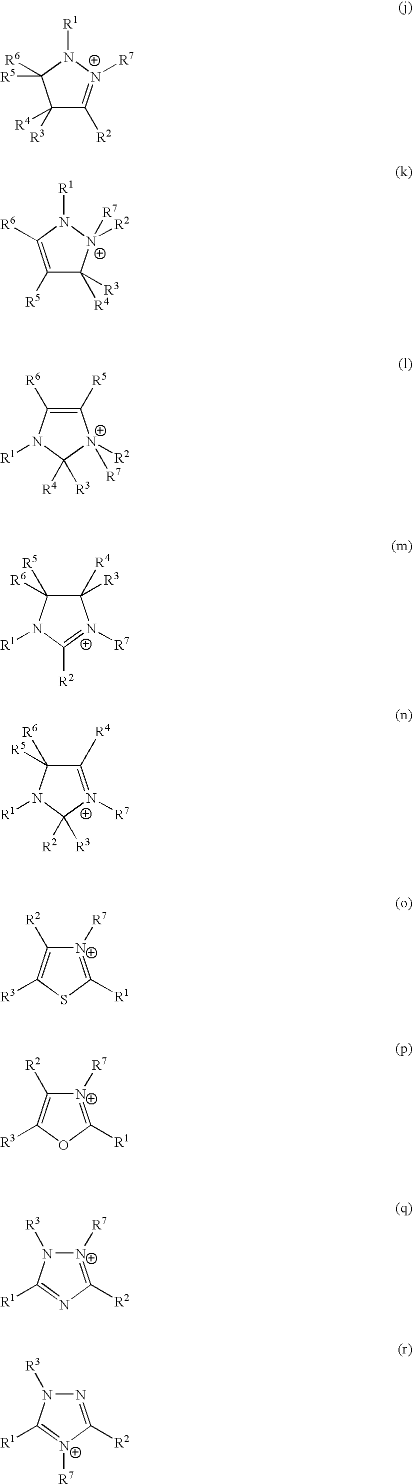 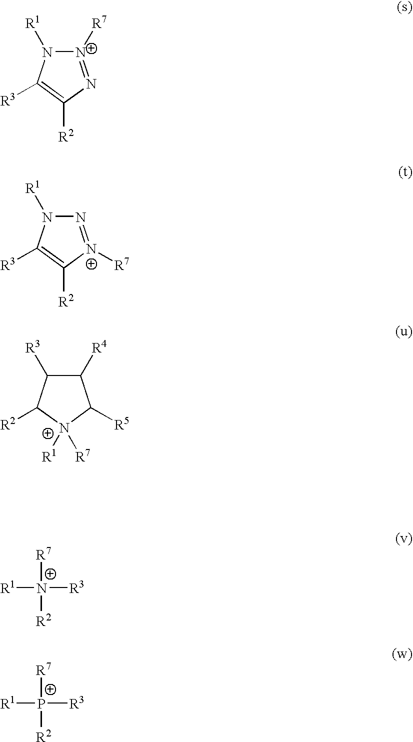
and also oligomers and polymers in which these structures are present,
where
R¹, R², R³, R⁴, R⁵, R⁶ and R¹ are each, independently of one another, hydrogen, C₁-C₁₈-alkyl, C₂-C₁₈-alkyl which may be interrupted by one or more oxygen and/or sulfur atoms and/or one or more substituted or unsubstituted imino groups, C₆-C₁₂-aryl, C₅-C₁₂-cycloalkyl or a five- or six-membered, oxygen-, nitrogen- and/or sulfur-containing heterocycle or two of them together form an unsaturated, saturated or aromatic ring which may be interrupted by one or more oxygen and/or sulfur atoms and/or one or more substituted or unsubstituted imino groups, where the radicals mentioned may each be, independently of one another, monosubstituted or polysubstituted by functional groups, aryl, alkyl, aryloxy, alkyloxy, halogen and/or heterocycles, with in the case of the ammonium ions (v), R¹, R², R³ and R⁷ not all being an unsubstituted alkyl radical,
R⁷ can also be C₁-C₁₈-alkyloyl, C₁-C₁₈-alkyloxycarbonyl, C₅-C₁₂-Cycloalkylcarbonyl or C₆-C₁₂-aryloyl, where the radicals mentioned may each be, independently of one another, monosubstituted or polysubstituted by functional groups, aryl, alkyl, aryloxy, alkyloxy, halogen and/or heterocycles and
functional groups are: carboxyl, carboxamide, hydroxyl, amino, C₁-C₄-alkylamino, di(C₁-C₄-alkyl)amino, C₁-C₄-alkyloxycarbonyl, cyano or C₁-C₄-alkyloxy,
and the anion is selected from the group consisting of:
halides, alkylcarboxylate, tosylate, sulfonate, dialkylphosphate, bis(trifluoromethylsulfonyl)imide, trifluoracetate, triflate, sulfate, hydrogensulfate, methylsulfate, ethylsulfate, sulfite, hydrogensulfite, chloroaluminates, bromoaluminates, nitrite, nitrate, chlorocuprate, phosphate, hydrogenphosphate, dihydrogenphosphate, carbonate and hydrogencarbonate,
is used.

7. A process as claimed in claim 1, wherein an ionic liquid which comprises one each of the following cations and anions,
where the cation is selected from the group consisting of:
1-methylimidazolium, 1-ethylimidazolium,-propylimidazolium, 1-butylimidazolium, 2-ethylpyridinium, 1-ethyl-3-methylimidazolium, 1-n-butyl-3-ethylimidazolium, 1,2-dimethylpyridinium, 1-methyl-2-ethylpyridinium, 1-methyl-2-ethyl-6-methylpyridinium, N-methylpyridinium, 1-butyl-2-methylpyridinium, 1-butyl-2-ethylpyridinium, 1-butyl-2-ethyl-6-methylpyridinium, N-butylpyridinium, 1-butyl-4-methylpyridinium, 1,3-dimethylimidazolium, 1,2,3-trimethylimidazolium, 1-n-butyl-3-methylimidazolium, 1,3,4,5-tetramethylimidazolium, 1,3,4-trimethylimidazolium, 1,2-dimethylimidazolium, 1-butyl-2,3-dimethylimidazolium, 3,4-dimethylimidazolium, 2-ethyl-3,4-dimethylimidazolium, 3-methyl-2-ethylimidazolium, 3-butyl-1-methylimidazolium, 3-butyl-1-ethylimidazolium, 3-butyl-1,2-dimethylimidazolium, 1,3-di-n-butylimidazolium, 3-butyl-1,4,5-trimethylimidazolium, 3-butyl-1,4-dimethylimidazolium, 3-butyl-2-methylimidazolium, 1,3-dibutyl-2-methylimidazolium, 3-butyl-4-methylimidazolium, 3-butyl-2-ethyl-4-methylimidazolium 3-butyl-2-ethylimidazolium, 1-methyl-3-octylimidazolium and 1-decyl-3-methylimidazolium,
and the anion is selected from the group consisting of:
halides, acetate, methanesulfonate, tosylate, sulfate, hydrogensulfate, phosphate, hydrogenphosphate, dihydrogenphosphate, dialkylphosphate and bis(trifluoromethylsulfonyl)imide,
is used.

8. A process as claimed in claim 1, wherein an ionic liquid selected from the group consisting of: 1-methylimidazolium chloride, 1-methylimidazolium bromide, 1-methylimidazolium fluoride, 1-methylimidazolium iodide, 1-methylimidazolium hydrogensulfate, 1-methylimidazolium sulfate, 1-methylimidazolium methanesulfonate, 1-methylimidazolium tosylate, 1-methylimidazolium diethylphosphate, 1-ethylimidazolium chloride, 1-ethylimidazolium bromide, 1-ethylimidazolium fluoride, 1-ethylimidazolium iodide, 1-ethylimidazolium hydrogensulfate, 1-ethylimidazolium sulfate, 1-ethylimidazolium methanesulfate, 1-ethylimidazolium tosylate, 1-ethylimidazolium diethylphosphate, 1-propylimidazolium chloride, 1-propylimidazolium bromide, 1-propylimidazolium fluoride, 1-propylimidazolium iodide, 1-propylimidazolium hydrogensulfate, 1-propylimidazolium sulfate, 1-propylimidazolium methanosulfate, 1-propylimidazolium tosylate, 1-propylimidazolium diethylphosphate, 1-butylimidazolium chloride, 1-butylimidazolium bromide, 1-butylimidazolium fluoride, 1-butylimidazolium iodide, 1-butylimidazolium hydrogensulfate, 1-butylimidazolium sulfate, 1-butylimidazolium methanesulfonate, 1-butylimidazolium tosylate, 1-butylimidazolium diethylphosphate, 2-ethylpyridinium chloride, 2-ethylpyridinium bromide, 2-ethylpyridinium iodide, 2-ethylpyridinium hydrogensulfate, 2-ethylpyridinium sulfate, 2-ethylpyridinium methanesulfonate, 2-ethylpyridinium tosylate, 2-ethylpyridinium diethylphosphate, 1-ethyl-3-methylimidazolium chloride, 1-ethyl-3-methylimidazolium bromide, 1-ethyl-3-methylimidazolium fluoride, 1-ethyl-3-methylimidazolium iodide, 1-ethyl-3-methylimidazolium hydrogensulfate, 1-ethyl-3-methylimidazolium sulfate, 1-ethyl-3-methylimidazolium methanesulfonate, 1-ethyl-3-methylimidazolium tosylate, 1-ethyl-3-methylimidazolium diethylphosphate, 1-n-butyl-3-methylimidazolium chloride, 1-n-butyl-3-methylimidazolium bromide, 1-n-butyl-3-methylimidazolium fluoride, 1-n-butyl-3-methylimidazolium iodide, 1-n-butyl-3-methylimidazolium hydrogensulfonate, 1-n-butyl-3-methylimidazolium sulfate, 1-n-butyl-3-methylimidazolium methanesulfonate, 1-n-butyl-3-methylimidazolium tosylate, 1-n-butyl-3-methylimidazolium diethylphosphate, 1-n-butyl-3-ethylimidazolium chloride, 1-n-butyl-3-ethylimidazolium bromide, 1-n-butyl-3-ethylimidazolium fluoride, 1-n-butyl-3-ethylimidazolium iodide, 1-n-butyl-3-ethylimidazolium hydrogensulfate, 1-n-butyl-3-ethylimidazolium sulfate, 1-n-butyl-3-ethylimidazolium methanesulfonate, 1-n-butyl-3-ethylimidazolium tosylate and 1-n-butyl-3-ethylimidazolium diethylphosphate,
is used.

9. A process as claimed in claim 8, wherein the ionic liquid is selected from the group consisting of: 1-methylimidazolium chloride, 1-methylimidazolium bromide, 1-methylimidazolium hydrogensulfate, 2-ethylpyridinium chloride, 2-ethylpyridinium bromide, 2-ethylpyridinium hydrogensulfate, 1-ethyl-3-methylimidazolium chloride, 1-ethyl-3-methylimidazolium bromide and 1-ethyl-3-methylimidazolium hydrogensulfate.

10. A process as claimed in claim 1, wherein the haloalkane is isolated from the reaction mixture by distillation.

11. A process as claimed in claim 1, wherein the ionic liquid has a melting point of less than 150° C.

12. A process as claimed in claim 11, wherein the melting point is less than 100° C.

13. A process as claimed in claim 1, wherein the reaction is carried out in the presence of from 1 to 3 mol of ionic liquid per mol of OH group to be reacted in the alcohol.

14. A process as claimed in claim 1, wherein the reaction is carried out in the absence of water or the substantial absence of water at the time of commencement of the.

15. A process as claimed in claims 1, wherein in the case of the reaction of alcohols having more than one OH group per molecule, all OH groups are replaced by halogen.

16. A process as claimed in claim 1, wherein the water liberated in the reaction is continuously removed.

17. A process as claimed in claim 16, wherein the water liberated in the reaction is distilled off.

18. A process as claimed in claim 16, wherein the water content in the reaction of the alcohol with the hydrogen halide is not more than 25 mol %, based on the amount of ionic liquid, over the entire reaction time.

19. A process as claimed in claim 18, wherein the water content is not more than 5 mol %, based on the amount of ionic liquid.
